(19) United States
(12) Patent Application Publication
(10) Pub. No.: US 2004/0037849 A1
(43) Pub. Date: Feb. 26, 2004

(54) Vaccines for cat scratch fever

(75) Inventors: Bruno B. Chomel, Davis, CA (US); Rickie W. Kasten, Davis, CA (US); Kazuhiro Yamamoto, Yao-City (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: REGENTS OF THE UNIVERSITY OF CALIFORNIA, Oakland, CA

(21) Appl. No.: 10/227,078

(22) Filed: Aug. 21, 2002

(51) Int. Cl. A61K 39/12; C12N 1/20

(52) U.S. Cl. 424/204.1; 435/252.1

(57) ABSTRACT

Vaccines for Cat Scratch Fever and methods of vaccinating a mammal against B. henselae are provided.

### BACKGROUND OF THE INVENTION

Cat Scratch Fever (also called Cat Scratch Disease or benign lymphoreticulosis) typically is manifested as a small skin lesion (resembling an insect bite) that develops at the site of a cat scratch or (less commonly) a bite, followed within two weeks by swollen lymph nodes and sometimes a fever. The illness is mild and self-limiting in the majority of patients, although it may take some months for the swollen lymph nodes to return to normal. Treatment is usually not required. Infection however may be associated with later development of such things as tonsillitis, encephalitis, hepatitis, pneumonia and other serious illnesses in some cases. People with compromised immune systems, such as AIDS and cancer patients, are most at risk and can become most seriously ill.

Domestic cats are the reservoir of Bartonella henselae, the main causative agent of Cat Scratch Disease (CSD) (Regnery, et al. Lancet 340, 557-558 (1992), Childs, et al. J. Am. Vet. Med. Assoc. 204, 1775-1778 (1994), Koehler, et al. JAMA. 271, 531-535 (1994), Chomel, et al. J. Clin. Microbiol. 33, 2445-2450 (1995), Yamamoto, et al. Vet. Immunol. Immunopathol. 65, 191-204 (1998), Breitschwerdt, et al. Clin. Microbiol. Rev. 13, 428-438 (2000)). This agent is also associated with various diseases, including endocarditis, osteomyelitis, neuroretinitis in immunocompetent persons, and bacillary angiomatosis, bacillary peliosis or endocarditis in immunocompromised individuals (Wong, et al. Clin. Infect. Dis. 21:352-360 (1995); Anderson, et al. Clin. Microbiol. Rev. 10:203-219 (1997); Maurin, et al. Eur. J Clin. Microbiol. Infect. Dis. 16:487-506 (1997); Reed, et al. Ophthalmology 105:459-466 (1998); Breitschwerdt et al., Clin. Microbiol. Rev. 13, 428-438 (2000); Chomel, B. B., Rev. Sci. Tech. Off. Int. Epizoot. 19:136-150 (2000); Koehler, J. E., BARTONELLA SPECIES (Nataro, J. P., M. J. Blaser and S. Cunningham-Rundles, Eds, 2000) pp., 339-353). Cat fleas (Ctenocephalides felis) are the main vectors of transmission among cats (Chomel, et al. J. Clin. Microbiol. 34:1952-1956 (1996); Higgins, et al. J. Med. Entomol. 33:490-495 (1996); Foil, L., et al. J. Med. Entomol. 35:625-628 (1998)). Based on 16S ribosomal RNA gene (rDNA) sequencing, B. henselae has been classified into two predominant serotypes/genotypes, type I and type II (Houston I and BA-TF/Marseille) (Drancourt, et al. Lancet 347:441-443 (1996); Bergmans, et al. J. Clin. Microbiol. 34:254-260 (1996); Bergmans, et al. J. Clin. Microbiol. 35:2256-2261 (1997); Heller, et al. J. Clin. Microbiol. 35:1327-1331 (1997); Sander, A., et al. J. Clin. Microbiol. 35:584-587 (1997); Sander, et al. J. Clin. Microbiol. 36, 2973-2981 (1998)).

Cats are also the reservoir of B. clarridgeiae, a potential zoonotic bacterium involved in CSD (Kordick, et al. J. Clin. Microbiol. 35:1813-1818 (1997); Margileth, et al. Clin. Infect. Dis. 27:353-357 (1998); Breitschwerdt et al., Clin. Microbiol. Rev. 13, 428-438 (2000); Chomel, B. B., Rev. Sci. Tech. Off. Int. Epizoot. 19:136-150 (2000); Sander, et al. J. Clin. Microbiol. 38:2943-2948 (2000)). Furthermore, cats can be simultaneously co-infected with B. henselae and B. clarridgeiae or by the two variants of B. henselae (Bergmans, et al. J. Clin. Microbiol. 34:254-260 (1996); Gurfield, et al. J. Clin. Microbiol. 35:2120-2123 (1997); Chomel, et al. Am. J. Trop. Med. Hyg. 60:593-597 (1999)).

Relapsing bacteremia has been reported in cats infected with B. henselae and B. clarridgeiae (Kordick, D. L., et al. J. Clin. Microbiol. 33:1655-1656 (1995); Yamamoto, et al. Vet. Immunol. Immunopathol. 65:191-204 (1998); Mikolajczyk, et al. Am. J. Vet. Res. 61:375-379 (2000))), suggesting possible antigenic variation. However, experimental infections of cats with B. henselae (Houston I) or B. koehlerae have never led to relapsing bacteremia.

Only a few studies have been conducted so far to determine the level of cross-protection between Bartonella species or between different strains of B. henselae. These experimental studies demonstrated that cats primarily infected with B. henselae could be protected from homologous re-infection (Greene, et al. J. Clin. Microbiol. 34:1682-1685 (1996); Regnery, et al. Am. J. Vet. Res. 57:1714-1719 (1996); Yamamoto, et al. Vet. Immunol. Immunopathol. 65:191-204 (1998)). However, cats challenged with a different species or different B. henselae types (type II followed by type I) showed bacteremia after the secondary infection (Yamamoto, et al. Vet. Immunol. Immunopathol. 65:191-204 (1998)).

Thus, there is a need in the art for a vaccine for preventing infection by a majority of strains of B. henselae. The present invention addresses these and other problems.

### BRIEF SUMMARY OF THE INVENTION

The present invention provides pharmaceutical compositions comprising a pharmaceutically acceptable excipient and an immunogenically effective amount of an attenuated or killed B. henselae Type I bacteria. In some embodiments, the pharmaceutical composition further comprises an adjuvant. In some embodiments, the adjuvant is selected from the group consisting of incomplete Freund's adjuvant, aluminum phosphate, aluminum hydroxide and alum. In some embodiments, the bacteria is attenuated. In some embodiments, the bacteria is killed. In some embodiments, the bacteria is isolated from a cat. In some embodiments, the bacteria is isolated from a human. In some embodiments, the pharmaceutical composition further comprises an immunogen that induces a response to a panleukopenia antigen, a feline herpesvirus I antigen, a feline calicivirus antigen, a leukemia virus antigen, or a rabies antigen.

The present invention also provides methods of inducing a protective immune response in a mammal against B. henselae or B. clarridgeiae. In some embodiments, the method comprising administering to the mammal a pharmaceutical composition comprising a pharmaceutically acceptable excipient and an immunogenically effective amount of an attenuated or killed B. henselae Type I bacteria. In some embodiments, the mammal is a cat. In some embodiments, the pharmaceutical composition is administered parenterally. In some embodiments, the method further comprises administering the pharmaceutical composition to the mammal at least three months after the initial administration of the pharmaceutical composition. In some embodiments, the pharmaceutical composition further comprises an adjuvant. In some embodiments, the adjuvant is selected from the group consisting of incomplete Freund's adjuvant, aluminum phosphate, aluminum hydroxide and alum. In some embodiments, the bacteria is attenuated. In some embodiments, the bacteria is killed. In some embodiments, the bacteria is isolated from a cat. In some embodiments, the bacteria is isolated from a human. In some embodiments, the pharmaceutical composition further comprises an immunogen that induces a response to a panleukopenia antigen, a feline herpesvirus I antigen, a feline calicivirus antigen, a leukemia virus antigen, or a rabies antigen.

Definitions

An “immunogenically effective amount” refers to an amount of an immunogen sufficient to induce a detectable humoral or cellular immune response in an animal.

An “attenuated” bacteria refers to a bacteria that is either unable to colonize a host, unable to cause disease in a host or causes significantly reduced disease symptoms in a host. Attenuated bacteria typically lack a genetic component involved in host colonization or pathogenicity.

A “protective immune response,” as used herein, refers to a cellular or humoral immune response that prevents or delays infection or disease caused by a specified pathogen.

### DETAILED DESCRIPTION OF THE INVENTION

I. Introduction

This invention provides vaccines comprising attenuated or killed B. henselae Type I bacteria. The present invention provides the surprising discovery that vaccination with B. henselae Type I bacteria provides protection from infection by B. henselae Type I or Type II.

The invention also provides methods of preventing infection or reducing the effects of infection by B. henselae Type I or Type II in a mammal by administering a vaccine comprising attenuated or killed B. henselae Type I bacteria. In some embodiments, the mammal is a cat (e.g., a house cat) or a human.

II. B. Henselae Type I Bacteria

The vaccines of the present invention comprise at least one strain of B. henselae Type I bacteria. Examples of Type I strains include those isolated from humans, e.g., Houston-1 [CIP 103737; G5436] (ATCC deposit number 49882) or strain AT7-66 (ATTC deposit number 49793); or those isolated from cats. Strains can be isolated using standard isolation techniques, typically from the blood of an infected individual. Isolated strains can then be “typed” using PCR amplification, RFLPs or other molecular methods known in the art to differentiate between Type I and Type II strains. See, e.g., Chomel et al., Vetinary Research 33:205-13 (2002); Bergmans, A. M., et al., J. Clin. Microbiol. 34, 254-260 (1996); Bergmans, A. M., et al. J. Clin. Microbiol. 35, 2256-2261 (1997).

In some embodiments, the bacteria are altered to reduce or eliminate pathogenicity. Development of attenuated bacteria strains is routine in the art and typically involves mutation of a gene or genes involved in pathogenesis or viability in a host. For example, U.S. Patent Publication No. 20020086032 and U.S. Pat. No. 6,254,874 describe methods of attenuating bacteria by inactivating gene products of bacteria. In cases where the art describes attenuation of different genera or species of bacteria, orthologs of genes from those genera or species can be isolated from B. henselae by standard molecular techniques and inactivated by, e.g., chemical mutagens, transposon tagging or homologous recombination.

Methods of making killed bacteria for vaccines are well known in the art. Bacteria can be killed, for instance, by heat, chemicals (e.g., formaldehyde or β-propyl lactone) or irradiation.

III. Formulation

The attenuated or killed B. henselae Type I bacteria of the invention may be combined or mixed with various solutions and other compounds as are known in the art. Vaccines may be prepared as injectables, as liquid solutions or emulsions. The bacterial strains of the invention may be mixed with pharmaceutically-acceptable excipients which are compatible with the bacterial strains. Excipients may include water, saline, dextrose, glycerol, ethanol, and combinations thereof. The vaccine may further contain auxiliary substances such as wetting or emulsifying agents, pH buffering agents, or adjuvants to enhance the effectiveness of the vaccines.

The vaccines of the invention will typically also comprise adjuvants. Examples of such adjuvants or agents include aluminum hydroxide, aluminum phosphate, aluminum potassium sulfate (alum), beryllium sulfate, silica, kaolin, carbon, water-in-oil emulsions, oil-in-water emulsions, muramyl dipeptide, bacterial endotoxin, lipid X, Corynebacterium parvum (Propionobacterium acnes), Bordetella pertussis, polyribonucleotides, sodium alginate, lanolin, lysolecithin, vitamin A, saponin, liposomes, levamisole, DEAE-dextran, blocked copolymers or other synthetic adjuvants. Such adjuvants are available commercially from various sources, for example, Merck Adjuvant 65 (Merck and Company, Inc., Rahway, N.J.) or Freund's Incomplete Adjuvant and Complete Adjuvant (Difco Laboratories, Detroit, Mich.). Other suitable adjuvants are Amphigen (oil-in-water), Alhydrogel (aluminum hydroxide), or a mixture of Amphigen and Alhydrogel.

The proportion of immunogen (i.e., attenuated or killed B. henselae Type I bacteria) and adjuvant can be varied over a broad range so long as both are present in effective amounts. For example, aluminum hydroxide can be present in an amount of about 0.5% of the vaccine mixture (Al₂O₃ basis). On a per-dose basis, the amount of the immunogen can range from about 5 μg to about 100 μg protein per patient. A preferable range is from about 20 μg to about 40 μg per dose. A suitable dose size is about 0.5 ml. Accordingly, a dose for intramuscular injection, for example, would comprise 0.5 ml containing 20 μg of immunogen in admixture with 0.5% aluminum hydroxide.

Conveniently, the vaccines are formulated to contain a final concentration of immunogen in the range of from 0.2 to 200 μg/ml, preferably 5 to 50 μg/ml, most preferably 15 μg/ml. After formulation, the vaccine may be incorporated into a sterile container that is then sealed and stored at a low temperature, for example 4° C., or it may be freeze-dried. Lyophilization permits long-term storage in a stabilized form.

IV. Administration

The vaccines of the invention may be administered by any conventional method for the administration of vaccines including oral and parenteral (e.g., subcutaneous or intramuscular) injection. The treatment may consist of a single dose of vaccine or a plurality of doses over a period of time. The immunogen of the invention (e.g., attenuated or killed B. henselae Type I bacteria) can be combined with appropriate doses of immunogens from other infectious agents.

Conventional protocols for immunizing a mammal can be used with the vaccines of the present invention. For parenteral administration, such as subcutaneous injection, examples of suitable carriers are the tetanus toxoid, the diphtheria toxoid, serum albumin and lamprey, keyhole limpet, or hemocyanin. Conjugates including these universal carriers can function as T cell clone activators in individuals having very different gene sets.

Oral formulations may include normally employed excipients such as, for example, pharmaceutical grades of saccharine, cellulose and magnesium carbonate. These compositions take the form of solutions, suspensions, tablets, pills, capsules, sustained release formulations or powders and contain 10-95% of the bacterial strains of the invention. Oral inoculation of cats is described in, e.g., Guptill L, et al. Vet Immunol Immunopathol. 71(3-4):233-43 (1999).

The peptides of this invention can be formulated for administration via the nasal passages. Formulations suitable for nasal administration, wherein the carrier is a solid, include a coarse powder having a particle size, for example, in the range of about 10 to about 500 microns which is administered in the manner in which snuff is taken, i.e., by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the active ingredient. Various ways of such administration are known in the art. The pharmaceutical formulation for nasal administration may be prepared as solutions in saline, employing benzyl alcohol or other suitable preservatives, absorption promoters to enhance bioavailability, fluorocarbons, and/or other solubilizing or dispersing agents known in the art. The unit dosage for nasal administration can be from 1 to 3000 mg, e.g., 10 to 1000 mg, or 1 to 10 mg of active ingredient per unit dosage form.

Vaccines of the invention may be combined with other vaccines for the same or other diseases to produce multivalent vaccines. A pharmaceutically effective amount of the immunogen can be employed with a pharmaceutically acceptable carrier such as a protein or diluent useful for the vaccination of mammals, particularly cats or humans. Other vaccines may be prepared according to methods well-known to those skilled in the art.

Examples of other vaccines, including other cat vaccines, that can be combined with attenuated or killed B. henselae Type I bacteria include, e.g., panleukopenia antigens, feline herpesvirus I antigens, feline calicivirus antigens, leukemia virus antigens, rabies antigens.

Those of skill will readily recognize that it is only necessary to expose a mammal to appropriate epitopes in order to elicit effective immunoprotection. The epitopes are typically segments of amino acids that are a small portion of the whole protein. Using recombinant genetics, it is simple and routine to alter a natural protein's primary structure to create derivatives embracing epitopes that are identical to or substantially the same as (immunologically equivalent) the naturally occurring epitopes. Such derivatives may include peptide fragments, amino acid substitutions, amino acid deletions and amino acid additions within the natural amino acid sequence for the select target protein. For example, it is known in the protein art that certain amino acid residues can be substituted with amino acids of similar size and polarity without an undue effect upon the biological activity of the protein.

### EXAMPLE

The following example is offered to illustrate, but not to limit the claimed invention.

The following example demonstrates that cats inoculated with B. henselae type I are protected against a heterologous challenge with B. henselae type II. The example also compares the characteristics of primary and secondary infections, such as duration of bacteremia, presence of bacteremia relapses, correlation between level of bacteremia and immune response by IFA in these cats.

Materials and Methods

Animals

Thirty-six 3-month to 2-year old SPF cats (20 males, 16 females), including 7 SPF cats from previous studies (Yamamoto, et al. Vet. Immunol. Immunopathol. 65, 191-204 (1998)), were enrolled in this study. Before experimental infection, all 36 cats were confirmed to be Bartonella spp. serologically and bacteriologically negative. All cats were examined clinically every day for the first two weeks and at least weekly thereafter. The cats were housed in groups of 4-6 (all infected with the same Bartonella species or type) in a controlled environment.

Experimental Inoculation

The Bartonella strains used for inoculation were respectively, B. henselae type I (either Houston I [ATCC strain #49882]: H1 or feline type I: F1), B. henselae type II, B. koehlerae (ATCC strain #700693), B. clarridgeiae (ATCC strain #51734). The B. henselae F1 and B. henselae type II strains were isolated from naturally infected cats and confirmed to be B. henselae by 16S rDNA sequencing (Gurfield, et al. J. Clin. Microbiol. 35, 2120-2123 (1997), Heller, et al. J. Clin. Microbiol. 35, 1327-1331 (1997)). These strains were grown either on blood agar plates (B. henselae and B. clarridgeiae) or chocolate agar (B. koehlerae), and incubated at 35° C. in 5% CO₂ (Yamamoto, et al. Vet. Immunol. Immunopathol. 65, 191-204 (1998); Droz, et al. J. Clin. Microbiol. 37:1117-1122 (1999)). The harvested colonies were suspended into sterile saline and 0.5 ml was inoculated intradermally in three to five different sites, as previously described (Abbott, et al. Comp. Immunol. Microbiol. Infect. Dis. 20:41-51 (1997)).

Of the 36 SPF cats, 16 cats were primarily infected with B. henselae type I with an inoculum of 6.64×10⁶ to 2.26×10⁸ CFU/ml (H1: 8 cats, F1: 8 cats); 10 cats were injected with B. henselae type II at a dose of 1.10×10⁶ to 9.60×10⁷ CFU/ml; six cats received a B. clarridgeiae inoculum of 1.00×10⁷ CFU/ml, and four cats were infected with B. koehlerae (inoculum dose: 8.60×10⁷ CFU/ml for 2 cats and 3.84×10⁸ CFU/ml for the 2 other cats). After the end of the initial bacteremic phase, when no colony counts could be observed on the agar plates, and after at least two months of successive negative cultures, these 36 cats were challenged with a heterologous strain as follows: 15 cats received B. henselae type I, (H1: 14 cats, F1: 1 cat) with an inoculum dose ranging from 3.04×10⁷ to 2.68×10⁸ CFU/ml, 13 cats were inoculated with B. henselae type II (inoculum doses: 1.00×1 to 7.40×10⁸ CFU/ml) and eight cats were re-infected with B. clarridgeiae (inoculum doses: 7.30×10⁶ to 1.08×10⁹ CFU/ml). The inoculum was confirmed to be B. henselae, B. clarridgeiae or B. koehlerae by polymerase chain reaction/restriction fragment length polymorphism (PCR/RFLP) analysis (Regnery, et al. J. Clin. Microbiol. 30:265-274 (1992), Gurfield, et al. J. Clin. Microbiol. 35:2120-2123 (1997), Droz, et al. J. Clin. Microbiol. 37:1117-1122 (1999)).

Blood Culture

Blood was drawn from the jugular vein of each cat weekly for the first month and every other week for the following months, up to 20 months after inoculation. Two ml of blood were placed into ethylenediamine tetraacetate (EDTA) tubes (Becton Dickinson, Franklin Lakes, N.J., USA) for culture and 1 ml into serum separation tubes for serological tests. The EDTA tubes were frozen at −70° C. and plated a few days later onto 5% rabbit blood agar plates for B. henselae and B. clarridgeiae and chocolate agar for B. koehlerae, as previously described (Abbott, et al. Comp. Immunol. Microbiol. Infect. Dis. 20:41-51(1997); Droz, et al. J. Clin. Microbiol. 37:1117-1122(1999)). The plates were incubated at 35° C. with 5% CO₂ for four weeks. Plates were examined 2-3 times a week for any bacterial growth. Isolated strains were confirmed to be Bartonella species by PCR/RFLP of the citrate synthase gene using HhaI and TaqI endonucleases. The number of colonies was counted and calculated as CFU/ml (Gurfield, et al. J. Clin. Microbiol. 35, 2120-2123 (1997); Droz, et al.. J. Clin. Microbiol. 37:1117-1122 (1999)). A bacteremia relapse was defined as any positive culture occurring after two successive negative cultures.

Immunofluorescent Antibody (IFA) Test

IgG antibody responses were detected by IFA, using antigens from the same strains used for inoculation (Childs, et al., J. Am. Vet. Med. Assoc. 204:1775-1778 (1994); Chomel, et al. J. Clin. Microbiol. 33:2445-2450 (1995)).

Statistical Tests

For duration of bacteremia, peak level, time to reach the maximum level of bacteremia, data were analyzed by one-way analysis of variance (ANOVA). For comparaison of peak levels of IgG antibodies, non-parametric Mann-Whitney rank sum test was performed with MINITAB™ statistical software Release 13.1 (MINITAB Inc., State Collage, Pa., USA). For peak level of bacteremia, equal variances t-test was performed with MINITAB™. For univariate analysis, non-parametric test (Fisher's exact test) was used for association between Bartonella species/types and bacteremia relapses (Epi-info version 6.04b, CDC Atlanta, Ga.). A p-value of <0.05 was considered as significant. Correlation between level of bacteremia and IFA titer was analyzed, using transformed linear regression analysis with MINITAB™.

Results

No obvious clinical signs were observed in the 28 cats inoculated with either B. henselae type I (H1), B. henselae type II, B. clarridgeiae or B. koehlerae. However, 6 of the 8 cats inoculated with B. henselae F1 developed fever (≧39.2° C.) within 2-12 days (mean: 5.8 days).

Primary inoculation: All 36 cats became bacteremic within one to two weeks after the primary inoculation, regardless of the Bartonella species or type inoculated (Table 1). Overall, bacteremia peaked between 14 and 52 days (median: 28 days) after primary inoculation (Table 2). No significant differences in time to reach the bacteremia peak was detected among cats primarily infected with either B. henselae type I, B. henselae type II, B. clarridgeiae or B. koehlerae (p>0.05). The maximum level of bacteremia ranged from 1.25×10² to 1.44×10⁶ CFU/ml (median 9.27×10⁴ CFU/ml) (Table 2) and no statistically significant differences between Bartonella species or types were detected (p>0.05). Duration of bacteremia in the 36 cats ranged from 37 days to 582 days (median: 98 days) (Table 2). Duration of bacteremia in the 16 cats inoculated with B. henselae type I was significantly shorter than for the 10 cats inoculated with either B. henselae type II (p=0.025) or the 6 cats inoculated with B. clarridgeiae (p=0.011). However, among the 16 cats infected with B. henselae type I, cats inoculated with B. henselae F1 had a longer duration of bacteremia than cats inoculated with either B. henselae H1 (p<0.001) or B. koehlerae (p=0.004). Similarly, cats inoculated with B. clarridgeiae showed a significantly longer duration of bacteremia than cats inoculated with B. koehlerae (p=0.01).

Challenge: Twenty-six of the 36 cats became bacteremic within one to two weeks after challenge (Table 1). Overall bacteremia peaked between 14 and 48 days (median: 27 days) after re-infection (Table 2). However, three of these 26 cats that were primarily inoculated with B. henselae type I and challenged with B. henselae type II had a transient and low bacteremia level (Table 1). No bacteremia was detected in three cats primarily inoculated with B. henselae F1 and challenged with B. henselae H1 (Table 1). Similarly, six of the 9 cats primarily inoculated with B. henselae type I and challenged with B. henselae type II did not develop any bacteremia, as shown in FIG. 1 for one of these cats. Among those nine cats, five (83%) of the six cats primarily inoculated with B. henselae H1 were protected from re-infection with B. henselae type II, whereas only one (33%) of the three cats primarily inoculated with B. henselae F1 was protected from re-infection. However, that difference was not statistically significant (p=0.222). Finally, one of the four cats primarily inoculated with B. clarridgeiae and challenged with B. henselae type I did not become bacteremic (Table 1). No significant differences in time to reach the bacteremia peak were observed among cats secondarily infected with either B. henselae type I, B. henselae type II or B. clarridgeiae (p>0.05).

TABLE 1
Experimental infection and re-infection of SPF cats with
various strains and species of Bartonella.

| | No. bacteremic | | No. bacteremic |
|---|---|---|---|
| | cats/ | | cats/ |
| Primary infection | No. inoculated | Challenge | No. inoculated |
| Strain | cats | Strain | cats |
| B. henselae type | 3/3 | B. henselae type I^{b} | 0/3 |
| I^{a} | | | |
| B. henselae type I | 9/9 | B. henselae type II | 3*/9 |
| B. henselae type | 6/6 | B. henselae type I | 6/6 |
| II | | | |
| B. henselae type I | 4/4 | B. clarridgeiae | 4/4 |
| B. henselae type | 4/4 | B. clarridgeiae | 4/4 |
| II | | | |
| B. clarridgeiae | 4/4 | B. henselae type I | 3/4 |
| B. clarridgeiae | 2/2 | B. henselae type II | 2/2 |
| B. koehlerae | 2/2 | B. henselae type I | 2/2 |
| B. koehlerae | 2/2 | B. henselae type II | 2/2 |
| Overall | 36/36 | | 26/36 |

*small number of colonies: mean 8.5 CFU/ml (range 2.6-17.0 CFU/ml)
^{a}B. henselae feline type I,
^{b}B. henselae Houston I

TABLE 2
Median days (and range) to reach peak bacteremia, median maximum bacteremia level (and range)
in CFU/ml and median duration in days (and range) of bacteremia in SPF cats infected and
challenged with various strains and species of Bartonella.

| | | | |
|---|---|---|---|
| Bartonella species/types | Median days to reach peak | Median maximum level of | Median duration in days of |
| (number of cats) | bacteremia (range) | bacteremia (CFU/ml) (range) | bacteremia (range) |

A. Primary Inoculation

| | | | | |
|---|---|---|---|---|
| B. henselae type I (n = 16) | 25 (14-48) | 9.33 × 10⁴ (1.25 × 10² − 4.45 × 10⁵) | 80 | (37-357) |
| B. henselae type II (n = 10) | 18 (14-52) | 9.27 × 10⁴ (2.40 × 10⁴ − 6.08 × 10⁵) | 181 | (49-582) |
| B. clarridgeiae (n = 6) | 36 (28-36) | 9.49 × 10⁴ (3.06 × 10³ − 3.20 × 10⁵) | 284 | (140-363) |
| B. koehlerae (n = 4) | 36 (14-36) | 7.12 × 10⁴ (3.36 × 10⁴ − 1.44 × 10⁶) | 74 | (140-363) |
| Overall (n = 36) | 28 (14-52) | 9.27 × 10⁴ (1.25 × 10² − 1.44 × 10⁶) | 98 | (37-582) |

B. Challenge

| | | | | |
|---|---|---|---|---|
| B. henselae type I (n = 11) | 28 (22-48) | 1.12 × 10⁴ (8.89 × 10⁰ − 9.36 × 10⁴) | 62 | (14-77) |
| B. henselae type II (n = 7) | 28 (14-35) | 3.00 × 10⁴ (2.67 × 10⁰ − 1.60 × 10⁵) | 70 | (37-203) |
| B. clarridgeiae (n = 8) | 22 (22-36) | 3.35 × 10⁴ (1.92 × 10³ − 8.69 × 10⁵) | 138 | (43-405) |
| Overall (n = 26) | 27 (14-48) | 9.27 × 10⁴ (2.67 × 10⁰ − 8.69 × 10⁵) | 63 | (14-405) |

Overall, peak levels of bacteremia in the 26 bacteremic challenged cats ranged from 2.67 CFU/ml to 8.69×10⁵ CFU/ml (median: 1.92×10⁴ CFU/ml) (Table 2) and these levels were significantly lower than those for primary inoculation (p=0.022). However, no significant differences in peak levels of bacteremia were observed among cats secondary infected with either B. henselae type I, B. henselae type II or B. clarridgeiae (p>0.10). In the 26 cats that were bacteremic after challenge, bacteremia lasted 14 to 405 days (median: 63 days) (Table 2). Overall, duration of bacteremia after challenge was significantly shorter than after primary inoculation (median: 98 days) (p=0.012). In particular, cats challenged with B. henselae (either type I or type II) had a significantly shorter duration of bacteremia (median: 63 days, n=18) than for cats primarily infected with B. henselae (median: 97 days, n=26) (p=0.013). Interestingly, only three (33%) of the nine cats primarily inoculated with B. henselae type I and challenged with B. henselae type II became bacteremic. Furthermore, these three cats had a significantly lower level (p<0.01) and shorter duration of bacteremia (p<0.05) when compared to any other groups of bacteremic cats after re-infection. Among the eight cats challenged with B. clarridgeiae, duration of bacteremia in the four cats primarily inoculated with B. henselae type I (median: 50 days) was significantly shorter than the duration of bacteremia in the four cats primarily inoculated with B. henselae type II (median: 284 days) (p=0.01). However, no statistically significant differences in the level of bacteremia were observed between these two groups (p>0.30).

Forty-four percent (n=16/36) of the primarily inoculated cats developed relapsing bacteremia compared to 27% (n=7/26) of the challenged bacteremic cats, but that difference was not statistically significant (p=0.16). During primary infection, cats inoculated with B. clarridgeiae were more likely to have relapses (100%; n=6/6) than the cats inoculated with either B. henselae type 1 (25%, n=4/16, p=0.002) or B. koehlerae (0%, n=0/4, p=0.03). Similarly, among challenged cats, the ones challenged with B. clarridgeiae were more likely to develop relapses (63%; n=5/8) than cats challenged with B. henselae (8%; n=2/25) (p=0.004). Cats primarily inoculated with B. henselae showed more relapses (38%; n=10/26) than cats heterologously challenged with B. henselae (11%; n=2/18) (p=0.03).

All thirty-six cats primarily infected with Bartonella sp. developed IgG antibodies, which were detected within 1-2 weeks (mean: 11 days) by IFA. Overall, IgG antibody titers peaked between 1 week and 21 weeks post-infection (PI) (mean: 6 weeks PI) and ranged from 1:512 to 1:8192 (mean: 1:1024). Positive IgG antibody titers (IFA titers≧1:64) persisted in all 36 cats for the duration of the study and no statistical differences were identified between each of the four Bartonella groups (p>0.11). Within each of the four Bartonella groups of primarily infected cats, a positive correlation (p<0.05) was observed between the level of bacteremia and the IFA titer for respectively 38% (n=6/16) of the cats inoculated with B. henselae type I, 20% (n=2/10) of the cats inoculated with B. henselae type II, 67% (n=4/6) of the cats inoculated with B. clarridgeiae and 25% (n=1/4) of the cats inoculated with B. koehlerae.

4. Discussion

Choosing appropriate strains of viruses or bacteria is of critical importance for the development of vaccines, especially for infectious agents with a wide diversity of strains (Gupta, S., et al. Science 280:912-915 (1998)). The present study is the first one to report protection of SPF cats by B. henselae type I (H1 or F1) from secondary infection by B. henselae type II. Similarly, we identified for the first time a reduction in the duration of bacteremia in SPF cats challenged with B. clarridgeiae after being primarily inoculated with B. henselae type I, whereas a similar effect was not observed for cats primarily infected with B. henselae type II. Previous studies on a limited number of cats have demonstrated the lack of re-infection after primary infection with a homologous strain of B. henselae (Greene, et al. J. Clin. Microbiol. 34:1682-1685 (1996), Regnery, et al. Am. J. Vet. Res. 57:1714-1719 (1996)). We have also shown protection in cats infected and re-infected with different isolates of B. henselae type II and we also identified a lack of cross protection in cats primarily infected with B. henselae type II and challenged with B. henselae type I (H1) or primarily infected with B. henselae type II and challenged with B. clarridgeiae.

We also confirmed that SPF cats inoculated with B. henselae type I and challenged with the same (H1) strain or a different strain (F1) of B. henselae type I did not become bacteremic. Despite a wide genomic diversity of B. henselae isolates as analyzed by pulsed-field gel electrophoresis (Sander, et al. J. Clin. Microbiol. 36:2973-2981 (1998), Maruyama, et al. Vet. Microbiol. 79:337-349 (2001), cats challenged with different strains of the same type of B. henselae (either type I or type II) were protected from re-infection. In contrast, 17 (94%) of the 18 cats challenged with a different Bartonella species became bacteremic. These observations support the lack of species cross-protection for feline Bartonella infection and only a unidirectional cross protection for B. henselae types (type I/type II, but not type II/type I). Our data are also supported by the fact that co-infection with B. henselae and B. clarridgeiae have been observed in naturally infected cats (Bergmans, et al. J. Clin. Microbiol. 35:2256-2261 (1997); Gurfield, et al. J. Clin. Microbiol. 35:2120-2123 (1997); Chomel, et al. Am. J. Trop. Med. Hyg. 60, 593-597 (1999)). Interestingly, natural coinfection by B. henselae type I and type II strains has also been reported (Gurfield, et al. J. Clin. Microbiol. 35:2120-2123 (1997)).

We confirmed that a longer duration of bacteremia in cats primarily inoculated with B. clarridgeiae was longer than in cats inoculated with either B. henselae type I or B. koehlerae (Yamamoto, et al. J. Clin. Microbiol. 40:466-474 (2002)). Furthermore, cats inoculated with B. henselae type II showed a longer duration of bacteremia than those inoculated with B. henselae type I. We also confirmed that cats inoculated with B. henselae type I (H1) had a significantly shorter duration of bacteremia than cats infected with B. henselae type I (F1).

Among the 36 challenged cats, twenty-six developed bacteremia. All but one cat challenged with a different Bartonella species became bacteremic. Among the 10 cats that did not become bacteremic, six were from a group of nine cats primarily infected with B. henselae type I and challenged with B. henselae type II. Furthermore, the three of these 9 cats that became bacteremic had a significantly lower bacteremia (mean; 8.5 CFU/ml) than for other infections.

Bacteremia was also significantly shorter in cats after challenge than after primary inoculation. Specifically, cats challenged with B. henselae (type I and II) had a shorter duration of bacteremia than cats primarily infected with B. henselae (type I and II). Cats challenged with B. clarridgeiae were more likely to have a shorter bacteremia when primarily infected with B. henselae type I than primarily infected with B. henselae type II. These observations suggest a possible cross protection against re-infection with B. clarridgeiae in cats primarily infected with B. henselae type I.

Cats primarily inoculated or challenged with B. clarridgeiae showed more relapses than cats inoculated with B. henselae. In previous studies, relapsing bacteremia has been observed in cats infected or re-infected with B. henselae and B. clarridgeiae, but the presence or absence of relapsing bacteremia in cats inoculated with B. henselae appeared to be associated with the inoculum strain used Kordick, et al. J. Clin. Microbiol. 33:1655-1656 (1995); Kordick, et al. Am. J. Vet. Res. 58:492-497 (1997), Guptill, et al. Vet. Immunol. Immunopathol. 65:177-189 (1998); Yamamoto, et al. Vet. Immunol. Immunopathol. 65:191-204 (1998)). For instance, cats inoculated with B. henselae type I (H1; a strain of human origin submitted to several passages), never presented relapsing bacteremia, whereas cats infected with feline B. henselae strains (either type I or type II) had relapses. Additionally, cats inoculated with B. henselae were more likely to develop relapses after primary inoculation than after challenge, suggesting induction of a partial protection.

IgG antibodies were detected by IFA in all 36 cats within a few weeks after primary inoculation. Cats experimentally infected with B. henselae, B. clarridgeiae or B. koehlerae develop specific IgG antibodies detectable by IFA within 1-3 weeks after infection. These antibodies can persist for several months, despite the absence of bacteremia. During primary infection with B. clarridgeiae, a positive correlation between bacteremia and IFA titers was observed in 67% of the cats, suggesting a limited role of the IgG antibody response in the organism defense mechanisms against Bartonella infection. Epidemiological and experimental studies of cats infected with Bartonella showed that longterm bacteremia was often concomitant with the presence of positive IFA titers. Similarly, most of the cats challenged with a heterologous strain of Bartonella developed bacteremia despite the presence of circulating antibodies. Therefore, the humoral immune response may not be sufficient to prevent Bartonella infection in cats.

In contrast to the humoral response, a predominant role of the cellular immune response has been established for many intracellular pathogens. Bartonella henselae can exist as an intracellular bacterium, located inside the host's erythrocytes and also has a tropism for endothelial cells. For cellular immunity, the major histocompatibility complex (MHC) class I pathway plays an important role for antigen presentation to CD 8⁺ T cells, but is not expressed on the surface of mammalian erythrocytes. Therefore, despite the presence of Bartonella organisms inside the erythrocytes, the cellular immune response, especially the CD8⁺ pathway, may not be triggered, leading to chronic Bartonella infection.

Immunopathological investigation of Bartonella infection in animal models is helpful for the development of vaccines. For feline infections, Pedersen, et al. Vet. Immunol. Immunopathol. 63, 83-103 (1998) demonstrated the essential role of CD4+ effector cells (Th1 and Th2) for activation of the immune system against intracellular bacteria. In a more recent study using a murine infection model with Bartonella, the importance of CD4+ Th1 T-cell dependent immune response leading to cell-mediated immune response was demonstrated. Similarly, in a canine model, dogs infected with B. vinsonii subsp. berkhoffii also developed cell-mediated immune response through CD4+ cells. Furthermore, immunocompromised humans infected with Bartonella, especially AIDS patients with very low CD4+ cell counts (<50cells/μl), present more severe clinical disease, such as bacillary angiomatosis. These observations clearly support the role of CD4+ cell-mediated immune pathway in Bartonella infections.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

What is claimed is:

1. A pharmaceutical composition comprising a pharmaceutically acceptable excipient and an immunogenically effective amount of an attenuated or killed B. henselae Type I bacteria.

2. The pharmaceutical composition of claim 1, wherein the pharmaceutical composition further comprises an adjuvant.

3. The pharmaceutical composition of claim 2, wherein the adjuvant is selected from the group consisting of incomplete Freund's adjuvant, aluminum phosphate, aluminum hydroxide and alum.

4. The pharmaceutical composition of claim 1, wherein the bacteria is attenuated.

5. The pharmaceutical composition of claim 1, wherein the bacteria is killed.

6. The pharmaceutical composition of claim 1, wherein the bacteria is isolated from a cat.

7. The pharmaceutical composition of claim 1, wherein the bacteria is isolated from a human.

8. The pharmaceutical composition of claim 1, wherein the pharmaceutical composition further comprises an immunogen that induces a response to a panleukopenia antigen, a feline herpesvirus I antigen, a feline calicivirus antigen, a leukemia virus antigen, or a rabies antigen.

9. A method of inducing a protective immune response in a mammal against B. henselae or B. clarridgeiae, the method comprising administering to the mammal a pharmaceutical composition comprising a pharmaceutically acceptable excipient and an immunogenically effective amount of an attenuated or killed B. henselae Type I bacteria.

10. The method of claim 9, wherein the mammal is a cat.

11. The method of claim 9, wherein the vaccine is administered parenterally.

12. The method of claim 9, further comprising administering the pharmaceutical composition to the mammal at least three months after the initial administration of the vaccine.

13. The method of claim 9, wherein the pharmaceutical composition further comprises an adjuvant.

14. The method of claim 13, wherein the adjuvant is selected from the group consisting of incomplete Freund's adjuvant, aluminum phosphate, aluminum hydroxide and alum.

15. The method of claim 9, wherein the bacteria is attenuated.

16. The method of claim 9, wherein the bacteria is killed.

17. The method of claim 9, wherein the bacteria is isolated from a cat.

18. The method of claim 9, wherein the bacteria is isolated from a human.

19. The method of claim 9, wherein the pharmaceutical composition further comprises an immunogen that induces a response to a panleukopenia antigen, a feline herpesvirus I antigen, a feline calicivirus antigen, a leukemia virus antigen, or a rabies antigen.
